(19) United States
(12) Patent Application Publication
(10) Pub. No.: US 2026/0094722 A1
(43) Pub. Date: Apr. 2, 2026

(54) COMPUTER-IMPLEMENTED METHOD FOR CLINICAL DECISION SUPPORT

(71) Applicant: Beckman Coulter, Inc., Brea, CA (US)

(72) Inventors: Scott Levin, Brea, CA (US); Jeremiah Hinson, Brea, CA (US)

(21) Appl. No.: 19/338,955

(22) Filed: Sep. 24, 2025

Related U.S. Application Data
(60) Provisional application No. 63/699,914, filed on Sep. 27, 2024.

(51) Int. Cl. G16H 50/30; G16H 10/60

(52) U.S. Cl. CPC G16H 50/30; G16H 10/60

(57) ABSTRACT

A computer-implemented method for clinical decision support includes obtaining, at a computing device, subject data associated with a subject, the subject data at least including: at least three vital parameters determined for the subject, and the age of the subject; and computing, based on the obtained subject data, at least one indicator indicative of the likelihood that the subject needs at least one critical emergency department intervention within a time period.

### CROSS-REFERENCE TO RELATED APPLICATIONS

This application claims priority to U.S. Provisional Application No. 63/699,914 entitled “COMPUTER-IMPLEMENTED METHOD FOR CLINICAL DECISION SUPPORT” filed on Sep. 27, 2024, the contents of which are incorporated herein in its entirety.

### TECHNICAL FIELD

The present disclosure generally relates to the field of clinical decision support. In particular, the present disclosure relates to a computer-implemented method for clinical decision support. The present disclosure also relates to a computing device configured to carry out steps of said method, to a corresponding computer program instructing the computing device to perform steps of said method, and to a computer-readable medium storing such computer program.

### BACKGROUND

Providing appropriate care for a patient after hospital admission is important. This in particular relates to patients with a broad range of pathology, from minor ailments to life-threatening illnesses. This variability in clinical severity, coupled with frequent interruptions, increasingly complex diagnostic data streams, and/or pervasive hospital overcrowding may make the rapid identification and treatment of patients with time-sensitive conditions difficult. These conditions have been associated with adverse outcomes for patients, clinicians and/or healthcare systems alike. These include poor therapeutic relationships and patient experience, increased risk for litigation, higher healthcare costs, and/or excess morbidity and mortality.

Tools that facilitate rapid and reliable indications regarding the patients' risks after hospital admission are desired. Specifically there is a desire to indicate/predict, at least to some extent, an appropriate or expected care need reliably and/or swiftly.

### SUMMARY

This is achieved by a computational/computer-implemented method and tool/system/computing device that allows for clinical decision support with respect to likelihood that the subject needs at least one critical emergency department intervention within a time period. More specifically, this is achieved by the subject matter of the independent claims. Exemplary embodiments are recited in the dependent claims and described in the following.

An aspect of the present disclosure refers to a computer-implemented method for clinical decision support, wherein the method comprises obtaining, at a computing device, subject data associated with a subject, the subject data comprising at least three vital parameters determined for the subject and the age of the subject. The method further comprises computing, based on the obtained subject data, at least an indicator indicative of the likelihood that the subject needs at least one critical emergency department intervention within a time period, and/or an indicator (risk threshold/score) indicative/predictive of (the appropriateness/needed/likelihood/recommendation/expectation for) a subject needing at least one critical emergency department intervention within a (e.g. specific or predetermined/preset) time period.

The present disclosure, therefore, may provide for an improved clinical decision support system, for example allowing to efficiently, reliably and accurately indicate the appropriateness/need/likelihood/recommendation/expectation for at least one critical emergency department intervention on a subject within a time period. Specifically, the critical intervention refers to the subject's stay in the ED, i.e. after ED admission.

For instance, aspects of the present disclosure may facilitate to objectively risk-stratify patients and anticipate the need for time-sensitive interventions in the emergency department (ED). Hence, this may may help to prevent adverse events in the emergency department. The present disclosure may provide for better predictability of the need for at least one critical emergency department intervention within a time period, when the subject is present in the ED. This may help to prioritize treatment of the subjects/patients in the ED.

In embodiments, the method may help to indicate the likelihood/risk/need for the critical intervention, so as to speed up the handling and/or treatment of the subject during ED stay. In some embodiments, the method may even help to indicate the likelihood/risk/need for the critical intervention, to prevent the situation requiring the critical intervention to occur at all.

The indicator refers to the ED, i.e. decisions to be made with respect to subjects staying in the ED. The indicator of the present disclosure may help in clinical decision making when the subject (patient) is present in the ED, e.g. while waiting in the ED for treatment.

The indicator may be seen as reflecting the risk, in the ED, that health conditions of the subject deteriorate accordingly during the time period and/or that the intervention will indeed be performed and/or the likelihood that similar patients have had an intervention within the time period in the past.

Patients may arrive at the ED of a hospital, and within the ED, triage may be performed. Triage may be the first clinical activity performed after ED arrival, and the first point where vital parameters are measured. Vital parameters may subsequently be measured during other parts of the ED stay as well, and may be repeated one to many times over the ED stay.

It is typically the case that subjects with severe health issues arrive at the ED of a hospital. Specifically for these subjects, the risk indication is important so as to prioritize the subjects waiting for treatment in an appropriate order, correctly reflecting the severeness of the individual health issue.

For the subject data, at least three vital parameters are determined for the subject. For example, the at least three vital parameters may first be measured at triage after arrival at the ED. Alternatively or additionally, the vital parameters are determined while staying in the ED.

If less than three vital parameters are measured and considered for computation of the indicator, the prediction may be less accurate. Preferable three vital parameters may be as follows: Heart rate, respiratory rate and body temperature.

The at least three vital parameters are three different vital parameters.

The age of the subject may specifically be relevant as it may help to specify at least one of the at least three vital parameters more clearly, namely with respect to age, in case of one or more age-dependent vital parameters.

It is emphasized that the computer-implemented method and device described herein relate to a data processing means for clinical decision support, and do not involve in and for itself any diagnostic or therapeutic activity. The disclosure may in particular be helpful in connection with the assessment of the likelihood that a subject needs at least one critical emergency department intervention within a time period. For example, it may help clinicians when they have to assess the health condition and the prospects of the subject.

As used herein, any parameter value of the subject or associated with the subject, for example any parameter value for the subject data, may be obtained based on a measurement, for example based on or using one or more samples from the patient, such as blood samples. This applies to any clinical data, subject data or patient data, such as demographics, vital signs, medical history and/or pertinent laboratory results, of the subject described herein as being usable to compute the at least one indicator.

It is emphasized that none of the parameter values or parameters referred to herein is limited to a particular type of measurement to obtain or measure the respective parameter value.

As used herein, determining the at least one indicator may include computing and/or assessing the likelihood or probability. Any reference herein to a single indicator optionally includes a plurality of indicators, unless stated otherwise. The same applies for any other indicators of the disclosure.

For instance, determining may generally relate to or include finding out or coming to a decision about appropriateness for at least one critical emergency department intervention within a time period, which may optionally include a calculation, a computation and/or a reasoning for the assessment of the indicator. Alternatively or additionally, computing the indicator may include determining based on mathematical means; algorithms and/or calculation the likelihood that the subject needs at least one critical emergency department intervention within a time period. In particular, the determination may be computer-aided, computer-assisted and/or computer-implemented.

As used herein, obtaining the subject data may comprise accessing said data and/or retrieving the subject data e.g. from the at least one memory of the computing device that carries out the method of the present disclosure, from the memory of another computing device, or from another remote data storage (a database, a secondary memory, a cloud storage or the like). Accordingly, in some cases, retrieving the subject data may comprise downloading said data. Additionally or alternatively, obtaining the subject data may comprise receiving said data, e.g. from a user or a computing device different from the computing device accessing the data. The two options are not mutually exclusive. For instance, obtaining the set of patient data may comprise receiving said set data, storing said set in the memory of the computer device and retrieving the set of patient data by accessing said memory. For instance, obtaining the subject data may comprise receiving the subject data at the computing device, for example at a data storage thereof. Alternatively or additionally, obtaining the subject data may include downloading, retrieving, and/or accessing the subject data by the computing device, for example at a data source remote from the computing device.

According to the method described herein, one or more indicators indicative of at least one critical emergency department intervention for a subject within a time period may be computed by the computing device based on the subject data. For instance, a single indicator may be computed. Alternatively or additionally, a plurality of indicators may be computed.

As used herein, the indicator may relate to a quantitative and/or qualitative measure indicative, representative and/or descriptive of the need of at least one critical emergency department intervention within a time period. For example, the indicator may include or refer to a numerical measure or score on arbitrary scale, for example between zero and 100 or any other scale, indicative of the appropriateness/need/likelihood/recommendation. Alternatively or additionally, the indicator may represent a particular likelihood level or tier for, such as low likelihood, medium likelihood, high likelihood.

A critical emergency department intervention may take place inside the emergency department (ED) and may be carried out e.g. by an acute/intensive care specialist. The need for the critical emergency department intervention may be an objective medical need, to avoid clinical deterioration (i.e. a temporal degradation to a less stable physiologic state).

A critical intervention is typically performed in the ED and addresses the acute need to maintain or improve the health of the subject/patient. It may be that the intervention itself is risky, but nevertheless increases the chances to survive. Specifically, a critical emergency department intervention is a critical intervention carried out in the emergency department.

A critical emergency department intervention may alternatively be defined as an intervention typically carried out in the emergency department to address an acute (physiological) need of a subject, e.g. during a health issue.

A critical emergency department intervention may be a life-saving intervention, otherwise the subject may die. The indicator of the present disclosure may reflect the likelihood that the subject will receive/undergo the intervention during the time period.

For the actual classification, the computing device may comprise a classifier, for example based on a Machine Learning model, as described in more detail hereinbelow.

By providing a classification result indicating indicator that, based on the classification result, a subject may have, interpretation of the at least one indicator may be simplified, and erroneous interpretation may be avoided, for example when compared to numerical results or scores.

A further aspect of the present disclosure refers to a computing device or system including one or more processors for data processing, wherein the computing device or system is configured to carry out steps of the method described herein.

A further aspect of the present disclosure refers to a computer program, which, when executed by one or more processors of a computing device or system, instructs the computing device or system to perform steps of the method described herein.

Yet a further aspect of the present disclosure refers to a computer-readable medium, for example a non-transitory computer-readable medium, storing such computer program.

Any disclosure presented herein with reference to one aspect of the present disclosure equally applies to any other aspect of the present disclosure, unless explicitly stated otherwise.

Optional features are detailed in the following:

According to an embodiment, the (preset, predetermined) time period corresponds to the time of the (physical) presence of the subject in the emergency department, i.e. an (estimated, e.g. maximum) duration of the stay of the subject in the emergency department. The time period may be 24 hours after arrival in the emergency department, more optionally 4 to 10 hours after arrival in the emergency department.

According to an embodiment, the method comprises computing, based on the obtained subject data, a time period prediction for the need for the critical emergency department intervention. This may allow for more specific predictions, which may be beneficial in terms of scheduling and overall patient health risk. Specifically, this may allow for a point in time at which the intervention will be needed to be calculated.

According to an embodiment, the subject data further includes static information on the subject's medical history since ED admission, information on the subject's demographic affiliation and/or information on the arrival mode of the subject. Static information refers to subject-related information which does not change after ED admission. Example arrival modes may be walking, by police, by ambulance, by medical flight, by wheelchair.

According to an embodiment, the subject data further comprises dynamic information on at least one medical symptom and/or complaint of the subject and/or on interventions undertaken at the subject since ED admission. Dynamic information refers to subject-related information which changes after ED admission.

According to an embodiment, the subject data further comprises at least one laboratory parameter determined for the subject. This may help to obtain a more complete picture that may help for more accurate calculation of the at least one indicator.

According to an embodiment, the critical emergency department intervention is selected among the group of critical emergency department interventions consisting of: airway and breathing support; electrical therapy; emergency medication; emergency procedure; hemodynamic support.

Specific examples are as follows: Airway and breathing support includes intubation and mechanical ventilation or non-invasive positive pressure ventilation. Electrical therapy includes defibrillation, emergent cardioversion, or external pacing. Emergency procedures include chest needle decompression, pericardiocentesis, or open thoracotomy. Hemodynamic support includes significant intravenous fluid resuscitation in the setting of hypotension, blood administration, or control of major bleeding. Emergency medications include naloxone, dextrose, atropine, adenosine, epinephrine, or vasopressors administration.

According to an embodiment, the indicator indicates the cumulative need of any intervention of the group of the critical emergency department interventions.

According to an embodiment, the indicator comprises at least two sub-indicators each individually indicating the need for at least one of the group of the critical emergency department interventions.

According to an embodiment, it is indicated what parameters relating to the subject have been taken into account as subject data for computing the at least one indicator. This may be seen as beneficial in terms of transparency. Specifically, the number and/or type of parameters, such as vital or laboratory parameters, may be indicated. Also, the value of the respective parameter may be indicated.

According to an embodiment, the subject data comprises at least one vital parameter trend for each of the at least three vital parameters determined for and measured at a plurality of points in time and/or at least one laboratory parameter trend for the at least one laboratory parameter determined for the subject and measured at a plurality of points in time. Additionally or alternatively, according to an embodiment, at least one information trend based on at least one dynamic information is determined for the subject and at a plurality of points in time.

Preferably, the subject data comprises at least three vital parameter trends, one trend per vital parameter of the at least three vital parameters, determined for and measured at a plurality of points in time.

A trend may allow for updating, e.g. continuously monitoring, of the parameters/information, during ED stay. The trend includes the parameter/information as a function of time. Updates on parameters/information may be provided once new clinical data is available, e.g. in the electronic health record (EHR). The trend may allow for real-time adaptability of the parameter/information and of the indicator calculated on this basis. This may help to determine a course over time as regards the parameters/information and, hence, of the indicator.

Optionally, the indicator trend may be based on the at least one laboratory parameter trend and/or the at least three vital parameters trend and/or the information trend. Additionally or alternatively, the indicator trend may be visualized, e.g. at a computing device, to further help in decision making.

According to an embodiment, the method includes computing an indicator trend comprising the at least one indicator computed for a plurality of points in time, optionally based on the at least one laboratory and/or vital parameter and/or information trend.

The indicator trend may allow to indicate a time-wise tendency of the indicator, e.g. compared to a previous (value of the) indicator. For example, the latest indicator may be more positive or more negative (in terms of abnormal vs. normal) than the previous indicator (value). An indicator trend may be visualized, e.g. by means of a timeline. About 3 to 10, optionally 5 to 10 updates per subject may be provided within the period of time, for at least one of the parameters and information, and, thus, the indicator.

Hence, values of the vital parameter and/or laboratory parameter and/or dynamic information over time may be included in the subject data. This may help to determine a course over time as regards parameters/information and, hence, an improved significance of the indicator.

According to an embodiment, the at least one laboratory parameter is selected among the group of parameters consisting of/comprising: MCHC value; Troponin value; Red blood cell count; White blood cell count; C-reactive protein value; Blood Urea Nitrogen value; Lymphocyte count or value; Hematocrit value; D-dimer value; Hemoglobin value; and RDW value. Other laboratory parameters are possible, so the group is not exhaustive.

According to an embodiment, the at least three vital parameters are selected among the group of parameters consisting of: body temperature; blood pressure; oxygen saturation; heart rate; and respiratory rate.

According to an embodiment, at least some of the at least three vital parameters are categorized in sub-categories, optionally according to age. Specifically, a sub-categorization of the vital parameters, in particular of at least some of the at least three vital parameters, is preferable. Hence, the sub-categories of vital parameters may allow for discrimination according to age. For example, blood pressure, heart rate, and respiratory rate may be seen as age-dependent vital parameters (as they are directly related to the heart and, thus, to its size). In particular for one or more of these three vital parameters, one or more sub-categories dependent on age may be defined.

According to an embodiment, further indicators are possible. According to an embodiment, at least two, optionally more (all) indicators are at least partially computed in parallel/simultaneously. Simultaneous computing of multiple indicators may be carried out for the sake of completeness, as it may turn out that one or more indicators are less decisive for the outstanding decision.

According to an embodiment, a critical care indicator and a hospitalization indicator are calculated, the critical care indicator indicative of in-hospitality mortality and/or need for admission to an intensive care unit within 24 hours after emergency department disposition, the hospitalization indicator indicative of hospital admission, disposition and/or hospital procedure within a predetermined time period. Separate, independent models and calculations may be used for these two indicators.

According to an embodiment, a composite risk score indicated by the critical care indicator and the hospitalization indicator is computed, wherein a lower risk score is indicated by the hospitalization indicator, and a higher risk score is indicated by the critical care indicator.

According to an embodiment, a composite indicator indicative of the (1) indicator indicative of the likelihood that the subject needs at least one critical emergency department intervention within a time period, the (2) critical care indicator and the (3) hospitalization indicator is calculated. The composite indicator may be regarded as a threshold to indicate a clinical risk level, as composite outcome. The composite indicator may indicate the composite outcome, e.g. by way of a risk score. This may help to classify the overall health risk. According to an embodiment, the composite indicator may indicate/predict/recommend the need/appropriateness/recommendation for critical care. Accordingly, the need for critical care may be a composite outcome comprised of the following three things (with outcome met if any one of the three occur): critical ED intervention, intensive care unit admission, and death.

According to an embodiment, computing the at least one indicator is based on at least one machine learning model, ML model, configured to receive and process input data associated with at least a subset of the subject data. The ML model may optionally provide as output the one or more indicators. For instance, the one or more indicators may be provided as classification result of the ML model.

According to an embodiment, determining the at least one indicator may include computing based on at least one machine learning model of the computing device configured to receive and process at least the subject data as input data.

According to an embodiment, the method includes collecting data from a health care database, optionally including electronic health record data of multiple database subjects, as input training data for training of the machine learning model, optionally wherein the health care database is specific to the subject for which the indicator is computed and/or specific to the emergency department in which the subject stays. In other words, the health care database may be specific to the emergency department in which the critical emergency department intervention is/will/would be performed on the subject. Optionally, the health care database may be specific to the healthcare (provider) organization (more optionally characteristics of the healthcare organization) of the emergency department in which the subject stays. Characteristics of the healthcare organization may e.g. include structural factors

The database and, thus, the trained model may depend on the patient population, i.e. the subject. For example, structural factors of the emergency department, such as number of beds or the technical equipment, may play a role whether a subject is admitted to the specific emergency department. Also the outcome may play a role, e.g. the discharge or disposition may depend on the hospital/ED, such as the remaining subject in the ED (who may tentatively be less or more sick). When using a dedicated database of the emergency department, the ED population at the specific hospital may be used for training of the model. In other words, retrospective EHR data may be used for training of the machine learning model. This may allow for individual training of the model and, thus, for more precise predictions, i.e. computation of the indicator.

The disclosure also relates to the individual machine learning model as such, and corresponding scope of protection. Specifically, the disclosure features a method for training a machine learning model includes collecting data from a health care database, optionally including electronic health record data of multiple database subjects, as input training data for training of the machine learning model, optionally wherein the health care database is specific to the subject and/or emergency department for which the indicator is computed.

According to an embodiment, the indicator is determined based on the at least one machine learning model of the computing device configured to compute the indicator based on obtaining at least three vital parameters and the age of the database subjects as the input training data.

According to an embodiment, the input training data is cleaned by excluding database subject data having less than two vital parameters, having no age and/or of pediatric database subjects. For example, data of subjects younger than 12 years may be excluded from input training data.

According to an embodiment, the input training data includes at least, per database subject, information on three vital signs, the age, a medical symptom and/or complaint, the database subject's demographic affiliation and/or an arrival mode of the database subject.

In particular, some aspects of the present disclosure can use a predictive machine learning (ML) model to analyze relevant clinical data and/or subject data, such as vital signs or values, hematology parameters and/or laboratory results of a subject, to compute the at least one indicator. The at least one machine learning model can, for example, comprise one or more of a trained logistic regression, a trained random forest, a trained gradient boosting algorithm, and a trained artificial neural network.

For example, the method may comprise determining the at least one indicator based on current subject data and past subject data, for example obtained within or over a time window of predetermined length. Alternatively or additionally, a predefined number of parameter values in the past may be considered, such as the last two, three, four or more parameter values of a particular parameter.

According to an embodiment, the method may include saving, optionally in an electronic patient file, at least a subset of the subject data as obtained and/or at least a subset of the at least one indicator as computed and/or at least the composite indicator as determined. The output may be (auto)generated as clinical note and/or may be saved in EHR. Put differently, the method may allow for integration in the EHR system.

According to an embodiment, the method comprises obtaining and/or determining contextual information associated with the determined at least one indicator. For example, the contextual information may include information or data indicative or reflective of a computational basis leading to the determined one or more indicators.

The contextual information may include data or information for interpreting the determined one or more indicators. Exemplary contextual information may include one or more notifications, messages or other informative means, which may optionally be provided at a user interface of the computing device. In an example, the contextual information may include an indication or information about the parameters and/or parameter values used for computing the one or more indicators, and optionally their contribution in the computation of the one or more indicators.

According to an embodiment, the method includes displaying, at a user interface, the at least one indicator of a plurality of subjects, and/or the at least one indicator trend determined for a subject at a plurality of points in time and/or at least the composite indicator as determined. This may help a clinician to have a better overview by means of visualization. This may even help to automatically reduces the overall patient(s) risk by way of the visualization.

In an exemplary implementation, the at least one machine learning model may be configured, for example trained, to receive, simultaneously or sequentially, the at least three vital parameters and the age, and to provide the indicator as output. Optionally, the indicator may be provided by the computing device along with other information, such as an uncertainty and/or contextual information for the indicator or its determination.

For instance, the computing device may be configured to invoke the at least one machine learning model upon receipt of at least a subset or the entire set of subject data. Therein, the at least one machine learning model may refer to a trained machine learning model or algorithm, for example trained with a set of training subject data. Accordingly, the training subject data may include the parameters the computing device or machine learning model is configured to process as inputs to compute the indicator, and an expected or desired output of the computing device or machine learning algorithm for one or more of the reference patients, such as for example an indicator for one or more reference subjects. The trained machine learning algorithm or model may, for example, be a trained gradient boosting algorithm, or any other artificial intelligence-based (AI-based) algorithm, such as an artificial neural network, a feed forward neural network and/or a deep neural network. Accordingly, the computer-implemented method described herein may at least partly be implemented as a trained machine learning algorithm or artificial intelligence-based algorithm. Alternative implementations of the method, for example based on a decision tree, however, are possible and also encompassed by the present disclosure.

According to an embodiment, the computing device comprises a classifier or classifier circuitry, which may include or be a trained machine learning algorithm or model, a trained gradient boosting algorithm, or any other AI-based algorithm, model implemented in the computing device in one or both hard-and software. The classifier may be part of a control circuitry of the computing device, which may include one or more processors for data processing or may be implemented as separate classifier circuitry in the computing device, for example in the form of an integrated circuit and/or application specific integrated circuit.

It should be noted that also more than two different machine learning models may be used. For example, a particular or dedicated machine learning model may be applied or invoked by the computing device to incorporate or consider historic patient data, for example indicative of a prior history of medical disease and/or treatment associated with the subject. Non-limiting examples of historic patient data may include data indicative of one or more of prior history or one or more surgeries performed on the subject or any other clinical or diagnostic data.

According to an embodiment, the computing device includes one or more machine learning models, for example implemented in a classifier circuitry or logic of the computing device, as described in more detail hereinabove and hereinbelow.

According to a second aspect of the present disclosure, there is provided a computing device or system including one or more processors for data processing, wherein the computing device is configured to carry out steps of the method according to the first aspect of the present disclosure, as described hereinabove and hereinbelow.

The computing device may optionally comprise a data storage, for example storing at least a part of the subject data, data derived therefrom, the at least one first indicator and/or information or data related thereto. Alternatively or additionally, software instructions or one or more computer programs may be stored at the data storage, which when executed by one or more processors of the computing device, may instruct the computing device to perform steps of the method according to the first aspect of the present disclosure, as described hereinabove and hereinbelow.

According to an embodiment, the computing device includes one or more machine learning models, for example implemented in a classifier circuitry or logic of the computing device, as described in more detail hereinabove and hereinbelow.

The computing device described herein may refer to any data processing device, including a standalone computing device or server and computing networks with a plurality of inter-operating computing devices, such as a cloud computing system or server system. Alternatively or additionally, the computing device may be embodied, at least in part, as mobile device, such as a smart phone a tablet computer, a notebook or the like.

The computing device may, in an example, comprise one or more communication interfaces configured to communicate with one or more remote devices, for example an external data storage or database, and/or one or more remote computing devices. For instance, at least a part of the subject data may be received via the communication interface from an external data storage. Alternatively or additionally, the computed at least one indicator or information related thereto may be transmitted to one or more remote devices or stored at the external data storage.

A third aspect of the present disclosure relates to a computer program, which, when executed by one or more processors of a computing device, instructs the computing device to perform steps of the method according to the first aspect of the present disclosure, as described hereinabove and hereinbelow.

A fourth aspect of the present disclosure relates to a computer-readable medium, for example a non-transitory computer-readable medium, storing a computer program, which, when executed by one or more processors of a computing device, instructs the computing device to perform steps of the method according to the first aspect of the present disclosure, as described hereinabove and hereinbelow. In particular, the computer-readable medium according to the fourth aspect of the present disclosure stores the computer program according to the third aspect of the present disclosure.

These and other aspects of the disclosure will be apparent from and elucidated with reference to the appended figures, which may represent exemplary embodiments.

### BRIEF DESCRIPTION OF THE DRAWINGS

The subject-matter of the present disclosure will be explained in more detail in the following with reference to exemplary embodiments which are illustrated in the attached drawings, wherein:

FIG. 1 shows a computing device or system for determining at least one indicator according to an exemplary embodiment;

FIG. 2 shows a flow chart illustrating a method of determining at least one indicator according to an exemplary embodiment.

The figures are schematic only and not true to scale. In principle, identical or like parts are provided with identical or like reference symbols in the figures.

### DETAILED DESCRIPTION OF EXEMPLARY EMBODIMENTS

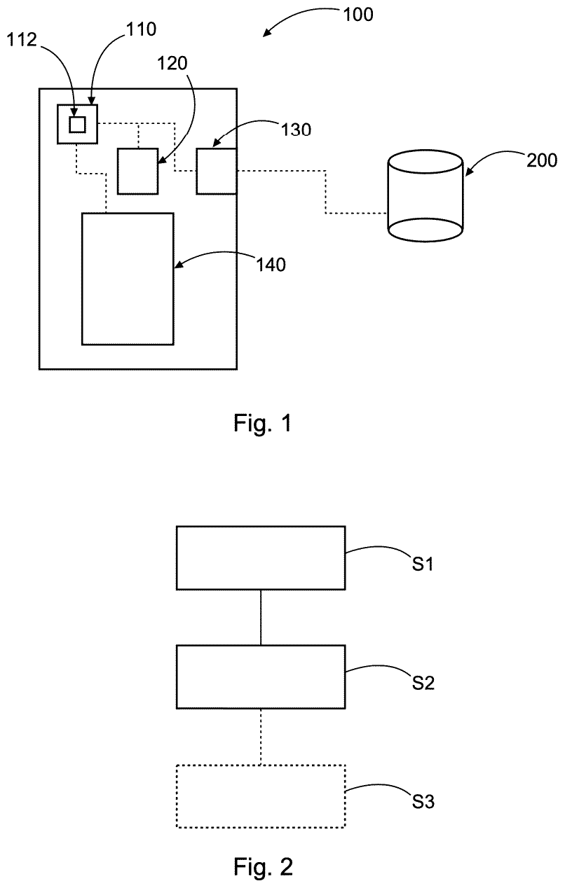

FIG. 1 shows a computing device or system 100, for example a clinical decision support system 100, configured to determine, for a subject (patient and/or individual), the at least one indicator indicative of at least one critical emergency department intervention.

The computing device 100 comprises a processing circuitry 110 or control circuitry 110 with one or more processors 112 for data processing. Optionally, the processing circuitry 110 or control circuitry 110 may include a classifier or a classifier circuitry. The computing device 100 further comprises at least one data storage 120 for storing data and/or software instructions. For example, at least a subset of the subject data and/or the at least one indicator may be stored at the data storage 120.

The exemplary computing device 100 of FIG. 1 further comprises at least one communication circuitry or interface 130 for communicatively coupling the computing device 100 to one or more external data sources 200 that may optionally store data and/or provide data to the computing device 100. The communication circuitry 130 may be configured for wired or wireless communication with the at least one external data source 200. It should be noted that the computing device 100 may comprise a plurality of communication circuits 130 or interfaces 130 for communicatively coupling the computing device 100 to a plurality of different external data sources 200.

The one or more external data sources 200 may for example be associated with one or more external servers communicatively coupled to the computing device 100, for example via the Internet, a LAN connection, a wireless connection or a wired connection. For example, the computing device 100 may be communicatively couplable to a hospital information system, a laboratory information system, a server of a health care provider, or any other server or data processing device.

As discussed in detail hereinabove and hereinbelow, the computing device 100 may be configured to perform steps of the method of determining the at least one indicator based on subject data including at least three vital parameters determined for the subject, and at least the age of the subject. Therein, the computing device 100 receives or obtains the subject data. Further, the computing device 100 is configured to determine, based on processing the received set of patient data, and/or compute the at least one indicator indicative/representative of (the appropriateness/need/likelihood/recommendation for) the subject's need for at least one critical emergency department intervention within a time period, as described in detail above.

The computing device 100 may be configured to carry out the method of any of the dependent claims and/or of the optionally features disclosed above.

In particular, the computing device 100 may be configured to compute the indicator for a time period corresponding to the (physical) presence of the subject in the emergency department and/or of 24 hours after arrival of the subject in the emergency department.

The computing device 100 may be configured compute, based on the obtained subject data, a time period prediction for the need for the critical emergency department intervention.

The computing device 100 may be configured to include in the subject data static information on the subject's medical history since ED admission, information on the subject's demographic affiliation and/or information on the arrival mode of the subject.

The computing device 100 may be configured to include in the subject data dynamic information on at least one medical symptom and/or complaint of the subject and/or on interventions undertaken at the subject since ED admission.

The computing device 100 may be configured to include in the subject data at least one laboratory parameter determined for the subject.

The computing device 100 may be configured to compute the indicator for a critical emergency department intervention selected among the group of critical emergency department interventions consisting of: airway and breathing support; electrical therapy; emergency medication; emergency procedure; hemodynamic support.

The computing device 100 may be configured to indicates the cumulative need of any intervention of the group of interventions or at least two sub-indicators each individually indicating the need for at least one of the group of interventions.

The computing device 100 may be configured to indicate what parameters relating to the subject have been taken into account as subject data for computing the at least one indicator.

The computing device 100 may be configured to include in the subject data at least one vital parameter trend for each of the at least three vital parameters determined for and measured at a plurality of points in time and/or at least one laboratory parameter trend determined for the subject and measured at a plurality of points in time.

The computing device 100 may be configured to include in the subject data at least one information trend based on at least one dynamic information determined for the subject and at a plurality of points in time.

The computing device 100 may be configured to compute an indicator trend comprising the at least one indicator computed for a plurality of points in time, optionally based on the at least one laboratory and/or vital parameter and/or information trend.

The computing device 100 may be configured to categorize at least some of the at least three vital parameters in sub-categories, optionally according to age.

The computing device 100 may be configured to compute a critical care indicator and a hospitalization indicator, the critical care indicator indicative of in-hospitality mortality and/or need for admission to an intensive care unit within 24 hours after emergency department disposition, the hospitalization indicator indicative of hospital admission, disposition and/or hospital procedure within a predetermined time period.

The computing device 100 may be configured to compute a composite risk score indicated by the critical care indicator and the hospitalization indicator, wherein a lower risk score is indicated by the hospitalization indicator, and a higher risk score is indicated by the critical care indicator.

The computing device 100 may be configured to compute a composite indicator indicative of the indicator indicative of the likelihood that the subject needs at least one critical emergency department intervention within a time period, the critical care indicator and the hospitalization indicator.

The computing device 100 may be configured to select the at least one laboratory parameter among the group of laboratory parameters consisting of: MCHC value; Troponin value; Red blood cell count; White blood cell count; C-reactive protein value; Blood Urea Nitrogen value; Lymphocyte count or value; Hematocrit value; D-dimer value; Hemoglobin value; and RDW value.

The computing device 100 may be configured to select the at least one vital parameter among the group of vital parameters consisting of: body temperature; blood pressure; oxygen saturation; heart rate; and respiratory rate.

The computing device 100 may be configured to compute the at least one indicator based on at least one machine learning model of the computing device configured to receive and process at least the subject data as input data.

The computing device 100 may be configured to collect data from a health care database, optionally including electronic health record data of multiple database subjects, as input training data for training of the machine learning model, optionally wherein the health care database is specific to the subject for which the indicator is computed.

The computing device 100 may be configured to determine the indicator based on the at least one machine learning model of the computing device configured to compute the indicator based on obtaining at least three vital parameters and the age of the database subjects as the input training data.

The computing device 100 may be configured to clean input training data by excluding database subject data having less than two vital parameters, having no age and/or of pediatric database subjects.

The computing device 100 may be configured to include in the input training data at least, per database subject, information on three vital signs, the age, a medical symptom and/or complaint, the database subject's demographic affiliation and/or an arrival mode of the database subject.

The actual computation of the at least one indicator may be performed based on at least one Machine Learning, ML, model of the computing device 100. The ML model may comprise one or more of a trained logistic regression, a trained random forest, a trained gradient boosting algorithm, and a trained artificial neural network.

Exemplarily, the ML model comprises the XGBoost algorithm defined by Chen and Guestrin. A benefit of XGBoost algorithm is that it can deal with missing information without requiring data imputation.

Also, it is noted that a plurality of any one or more of the aforementioned parameters may be comprised in the subject data and further parameter values may be derived therefrom by the computing device. In particular, the computing device 100 may be configured to determine a minimum diastolic blood pressure value among a plurality of diastolic blood pressure values and compute the at least one indicator based thereon. Alternatively or additionally, the computing device 100 may be configured to determine a minimum respiratory rate value among a plurality of respiratory rate values and compute the at least one first indicator based thereon. Alternatively or additionally, the computing device 100 may be configured to determine a minimum body temperature value among a plurality of body temperature values and compute the at least one indicator based thereon.

The computing device 100 may be configured to save, optionally in an electronic patient file, at least a subset of the subject data as obtained and/or at least a subset of the indicators as computed.

The computing device 100 may be configured to display, at a user interface, the at least one indicator of a plurality of subjects, and/or the at least one indicator trend determined for a subject at a plurality of points in time.

FIG. 2 shows a flow chart illustrating a computer-implemented method of determining at least one indicator according to an exemplary embodiment, for example using a computing device 100 as described with reference to FIG. 1.

Step S1 comprises obtaining, at a computing device 100, subject data associated with a subject after ED arrival, e.g. at the triage. The subject data comprises a) at least three vital parameters determined for the subject, and b) at least the age of the subject.

Step S2 comprises computing, based on the obtained subject data with the computing device 100, at least one indicator indicative of the subject's need for at least one critical emergency department intervention within a time period.

It is noted that one or more further optional steps, as described hereinabove in the summary part and with reference to FIG. 1, may be performed for determining the at least one indicator and/or for further steps/features according to the disclosure.

For example, in an optional step S3, the subject's need for at least one critical emergency department intervention within a time period may be displayed at the user interface 140 of the computing device 100.

Further optional steps according to the optional features, e.g. as defined in the dependent claims and/or as disclosed herein above, may be carried out.

Optionally, the computing device 100 may include one or more machine learning algorithms or models configured to process at least a part of the obtained set of subject data and compute the indicator.

In particular, a plurality of machine learning models may be implemented at the computing device 100, which may be invoked in dependence of the parameters or subject values of the set of subject data available.

The EHR data may further include an indication about one or more diseases a patient suffered from in the past. For instance, diagnosis codes, such as according to the International Statistical Classification of Diseases and Related Health Problems, so-called ICD codes, can be stored in the EHR data. Based on the diagnosis codes, it may be determined whether an issue occurred in a particular subject in the past, such that a corresponding label for the indicator can be generated and included in the training patient data.

The training patient data obtained from the EHR data may optionally be cleaned for subsequent processing, for example based on removing erroneous data records. Further optionally, the training patient data may be split into a training dataset, which can be used for optimizing the one or more machine learning models, and a test dataset, which can be used for validation of the one or more machine learning models. For instance, model parameters of the one or more machine learning models can be tuned or adjusted using the training dataset to determine optimized model parameters, which may then be used to validate the one or more machine learning models using the test dataset. The process may be repeated iteratively, for example until the one or more machine learning models satisfy a target performance on the test dataset.

In the following, an exemplary implementation of a machine learning model and aspects of training such model are summarized. The machine learning model may be based on or be a gradient boosting algorithm, but any other artificial intelligence-based (AI-based) algorithm, such as an artificial neural network, a feed forward neural network and/or a deep neural network, can be used instead.

As mentioned above, the at least one indicator may be computed based on or using one or more ML models. The one or more machine learning models may be trained using a set of historical emergency department (ED) electronic health record (EHR) data from health care systems, e.g. across the USA, as training data (also referred to as training subject data). An inclusion criterion can be that the ED/EHR data include at least one vital parameter and one laboratory parameter value. The data may include de-identified demographic, clinical, and diagnostic information for the patient or subject in the encounter.

For example, the training data or training subject data may be obtained from the EHR data based on extracting data being associated with a particular subject and being indicative of one or more of the parameters considered as input for the one or more machine learning models, such as any parameter described herein to be potentially usable for computing the at least one indicator. Each instance of patient or EHR data in the training data or training subject data may be associated with a respective known outcome in order to label the training data. For instance, the label can be a binary label wherein label 1 may equal or indicate a multiple overnight stay and label 0 may indicate a single night hospitalization, or vice versa. In an example, the ML model can be trained to provide the likelihood of subject data provided as input data being associated with the need for critical emergency department intervention in a time period.

The EHR data may further include an indication about one or more diseases a patient suffered from in the past. For instance, diagnosis codes, such as according to the International Statistical Classification of Diseases and Related Health Problems, so-called ICD codes, can be stored in the EHR data.

The training subject data obtained from the EHR data may optionally be cleaned for subsequent processing, for example based on removing erroneous data records. Further optionally, the training subject data may be split into a training dataset, which can be used for optimizing the one or more machine learning models, and a test dataset, which can be used for validation of the one or more machine learning models. For instance, model parameters of the one or more machine learning models can be tuned or adjusted using the training dataset to determine optimized model parameters, which may then be used to validate the one or more machine learning models using the test dataset. The process can be repeated iteratively, for example until the one or more machine learning models satisfy a target performance on the test dataset.

In the following, an exemplary implementation of a machine learning model and aspects of training such model are summarized. The machine learning model may include one or more of a trained logistic regression, a trained random forest, a trained gradient boosting algorithm, and a trained artificial neural network.

In particular, the method described herein may be implemented at least in part in a logistic regression ML model. Alternatively, a boosting technique may be utilized, such as XGBOOST, which however, is optional only.

When trained and/or during inference of the trained ML model, the at least one vital parameter and the at least one laboratory parameter of a subject can be provided as input. The at least one first indicator may then be computed and a corresponding output may be generated, for example in the form of %-appropriateness for multiple overnight hospitalization.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A computer-implemented method for clinical decision support, the method comprising:
obtaining, at a computing device, subject data associated with a subject, the subject data at least comprising:
a) at least three vital parameters determined for the subject, and
b) the age of the subject; and
computing, based on the obtained subject data, at least one indicator indicative of the likelihood that the subject needs at least one critical emergency department intervention within a time period.

2. The method of claim 1, wherein the time period is during the presence of the subject in the emergency department and/or within 24 hours of arrival to the emergency department.

3. The method of claim 1, further comprising computing, based on the obtained subject data, a time period prediction for the need for the critical emergency department intervention.

4. The method of claim 1, the subject data further comprising:
(c) static information on the subject's medical history since ED admission, information on the subject's demographic affiliation and/or information on the arrival mode of the subject.

5. The method of claim 4, the subject data further comprising:
d) dynamic information on at least one medical symptom and/or complaint of the subject and/or on interventions undertaken at the subject since ED admission.

6. The method of claim 4, the subject data further comprising:
d) at least one laboratory parameter determined for the subject.

7-9. (canceled)

10. The method of claim 1, wherein it is indicated what parameters relating to the subject have been taken into account as subject data for computing the at least one indicator.

11. The method of claim 1, the subject data comprising at least one vital parameter trend for each of the at least three vital parameters determined for and measured at a plurality of points in time and/or at least one laboratory parameter trend determined for the subject and measured at a plurality of points in time.

12. The method of claim 1, the subject data comprising at least one information trend based on at least one dynamic information determined for the subject and at a plurality of points in time.

13. The method including computing an indicator trend comprising the at least one indicator computed for a plurality of points in time.

14. The method of claim 1, wherein the at least one laboratory parameter is selected among the group of laboratory parameters comprising:
MCHC value;
Troponin value;
Red blood cell count;
White blood cell count;
C-reactive protein value;
Blood Urea Nitrogen value;
Lymphocyte count or value;
Hematocrit value;
D-dimer value;
Hemoglobin value; and
RDW value.

15. The method of claim 1, wherein the at least three vital parameters are selected among the group of vital parameters consisting of:
body temperature;
blood pressure;
oxygen saturation;
heart rate; and
respiratory rate.

16. (canceled)

17. The method of claim 1, wherein determining the at least one indicator includes computing based on at least one machine learning model of the computing device configured to receive and process at least the subject data as input data.

18. The method according to claim 17, wherein the method includes collecting data from a health care database, including electronic health record data of multiple database subjects, as input training data for training of the machine learning model, wherein the health care database is specific to the subject for which the indicator is computed and/or specific to the emergency department in which the subject stays.

19. The method according to claim 18, wherein the indicator is determined based on the at least one machine learning model of the computing device configured to compute the indicator based on obtaining at least three vital parameters and the age of the database subjects as the input training data.

20-21. (canceled)

22. The method of claim 1, wherein a critical care indicator and a hospitalization indicator are computed, the critical care indicator indicative of in-hospitality mortality and/or need for admission to an intensive care unit within 24 hours after emergency department disposition, the hospitalization indicator indicative of hospital admission, disposition and/or hospital procedure within a predetermined time period.

23. The method of claim 22, wherein a composite risk score indicated by the critical care indicator and the hospitalization indicator is computed, wherein a lower risk score is indicated by the hospitalization indicator, and a higher risk score is indicated by the critical care indicator.

24. The method of claim 22, wherein a composite indicator indicative of the indicator indicative of the likelihood that the subject needs at least one critical emergency department intervention within a time period, the critical care indicator and the hospitalization indicator is computed.

25. A computing device including one or more processors for data processing, wherein the computing device is configured to carry out the method according to claim 1.

26. (canceled)

27. A non-transitory computer-readable medium storing a computer program which, when executed by one or more processors of a computing device, instructs the computing device to perform the method according to claim 1.
